(19) United States
(12) Patent Application Publication
(10) Pub. No.: US 2026/0160342 A1
(43) Pub. Date: Jun. 11, 2026

(54) STOPCOCK AND PLUG FOR A STOPCOCK AND METHOD OF MANUFACTURING A PLUG FOR A STOPCOCK

(71) Applicant: OLYMPUS WINTER & IBE GMBH, Hamburg (DE)

(72) Inventors: Hannes MIERSCH, Hamburg (DE); Volker BLANKE, Hamburg (DE)

(73) Assignee: OLYMPUS WINTER & IBE GMBH, Hamburg (DE)

(21) Appl. No.: 19/323,792

(22) Filed: Sep. 9, 2025

(30) Foreign Application Priority Data
Sep. 11, 2024 (DE) 10 2024 126 047.5

(51) Int. Cl. F16K 5/02; B23P 23/02

(52) U.S. Cl. CPC F16K 5/0207; B23P 23/02; F16K 5/0242

(57) ABSTRACT

A plug for a stopcock as well as a stopcock and a method for manufacturing a plug, by means of which the aforementioned problems are solved. This is achieved by the fact that the plug, consisting of a cone part and a handle part, is formed in one piece. This plug can be rotatably mounted in an interior of a stopcock housing and has the cone part, which has a conical surface, and the handle part, which has a handle, lying one behind the other in the direction of an axis of rotation. The conical part also has a bore perpendicular to the axis of rotation, which can be designed as a fluid channel, whereby the conical surface can be brought into a sealing connection with an inner surface of the interior of the housing. The cone part is manufactured together with the handle part from a single workpiece by means of a turning and milling process.

The invention relates to a plug for a stopcock according to claim 1 and the general term of claim 8. Furthermore, the invention relates to a stopcock according to claim 12. Finally, the invention also relates to a method for manufacturing a plug for a stopcock according to the claim 5 and claim 13.

Medical instruments, such as endoscopes, usually have an elongated, tube-like shaft and optics that are used to examine the interior of the human body and for minimally invasive procedures. Through the shaft section of the instrument, through-hole instruments such as stone traps, instruments for electrosurgical resection or forceps can be guided to the surgical site. In order to flush away any local bleeding that occurs during the procedure and to protect the tissue from heat damage caused by high-frequency electrosurgical application, for example, the instruments are often equipped with a flushing device that permanently flushes the tissue located in front of the distal end of the shaft. The irrigation fluid can be fed through separate fluid channels through an inner tube or through a sheath tube of the instrument. In order to be able to control the inflow and outflow of the fluid, the fluid channels usually have a stopcock in their proximal end area. Corresponding stopcocks are described, for example, in DE 10 2014 002 158 B4 and DE 10 2016 011 184 A1.

The stopcocks usually consist of a housing and a rotating plug. The housing usually has a cuboid or cylindrical basic shape with an interior. This interior can be connected via two connecting pchiarts to a fluid channel of the instrument and, for example, to a supply line of a pump or similar. The plug can be rotationally fixed in this particularly conical interior. The plug consists of a conical part with a conical surface, whereby the shape of the conical part essentially corresponds to the shape of the interior of the housing. The plug also has a handle part with a grip. This handle, which is connected to the cone part, allows the plug to be rotated about an axis of rotation. The cone part has a hole running through it, which is usually perpendicular to the axis of rotation. The dimensions and orientation of this hole correspond to the openings of the two connecting parts in the housing. By rotating the plug around the axis of rotation, the bore with its two openings can be aligned with the openings of the two connection parts of the housing so that a continuous channel is formed through the stopcock. A further corresponding rotation of the plug can bring the hole into a position in which the continuous channel or the fluid channel is interrupted. In both positions, it is of crucial importance for the functionality of the stopcock that the fluid only flows through the channel and not between the conical surface and the inner wall of the housing or that no fluid flows through the stopcock.

The conical surface of the plug has ridges for this sealing effect of the stopcock and for channeling the fluid. These webs are arranged as protrusions on the conical surface and are designed in such a way that they seal with the inner wall of the housing. For this purpose, two ring-like webs can be assigned to the cone part above and below the bore. Due to the conical shape of the conical part, these ring-like webs have different diameters. Furthermore, vertical webs can be formed on the conical surface, which are arranged laterally at the openings of the bore and extend from one ring-like web to the other ring-like web.

In order to achieve the sealing effect of the stopcock, these bars must be very precisely designed or manufactured and also require a high degree of material hardness for appropriate durability. As a rule, the plugs are made of wear-resistant and dimensionally stable materials such as PEEK. In order to achieve the precision of the sealing surfaces, the PEEK material is machined in a turning process. This turning process takes place on the semi-finished PEEK material or after the PEEK component or the cone part of the plug has been produced in a previous injection molding process. Once the sealing surfaces of the cone part have achieved sufficient precision through the turning process, the handle part of the plug is bonded to the cone part of the plug in a further process step. Afterwards, the multi-part plug, which has been produced in several process steps, must be thoroughly cleaned.

If the plug is injection-molded together with the handle, it is necessary for the finished plug to be picked up in another machine for turning after injection-molding. The sufficient precision of the sealing surfaces of the bars is only achieved by this further process step of overmolding. Both of the manufacturing processes outlined here for a plug for a stopcock are very complex and cost-intensive.

On this basis, the present invention is based on the problem of creating a plug for a stopcock as well as a stopcock and a method for manufacturing a plug, by means of which the aforementioned problems are eliminated.

A solution to the said problem is described by the features of claim 1. Accordingly, it is provided that the plug, consisting of a cone part and a handle part, is formed in one piece. This plug can be rotatably mounted in an interior of a housing of a stopcock and has the cone part, which has a conical surface, and the handle part, which has a handle, lying one behind the other in the direction of an axis of rotation. The conical part also has a bore perpendicular to the axis of rotation, which can be designed as a fluid channel, whereby the conical surface can be brought into a sealing connection with an inner surface of the interior of the housing. The cone part is manufactured together with the handle part from a single workpiece by means of a turning and milling process. This combined process in a corresponding machine, preferably a CNC machine, allows the plug to be manufactured in one piece, eliminating the need to join individual components. In addition, the turning and milling process allows the necessary precision to be achieved, which is required for the tightness of the plug or stopcock. This one-piece production of the plug using the combined turning and milling process means that the plug can be produced in a particularly time and cost-efficient manner. At the same time, the necessary precision can also be achieved.

Preferably, the invention further provides that the shape of the cone part and the shape of the handle part are reduced to essential shapes and features so that the plug can be manufactured in one piece by the combined turning and milling process. This preparatory or adapted design makes it possible to manufacture the plug in one piece. Accordingly, the shapes are selected in such a way that they are compatible with both the turning process and the milling process. It is conceivable that more complex structures of the cone surface are dispensed with in order to make the cone surface simple, which also simplifies the turning process. It is conceivable that the more complex features, such as webs, which contribute to a sealing effect between the cone part and the housing, could be moved into the housing. This design of the plug, in particular the cone part and the handle part, contributes significantly to making the production of the plug particularly time- and cost-efficient. Accordingly, a cone with a corresponding cone surface with the bore or the fluid channel could be regarded as the essential shape of the cone part. At least one, preferably two, surfaces on the plug or the cone part that are suitable for rotating actuation of the plug could be designated as the essential shape of the handle part.

A particularly advantageous embodiment of the invention may provide for the plug to be made of polyoxymethylene (POM). POM is particularly advantageous because it is a thermoplastic with high mechanical strength and rigidity. POM also has very good sliding properties and high wear resistance. POM also absorbs very little moisture. Another advantage of POM is that it is recyclable. POM enables an excellent surface quality after processing, which is particularly important for plugs. These properties make the material particularly suitable for the production of plugs.

Preferably, it is further conceivable that the cone surface has at least two horizontal webs which extend in a ring-like manner around the cone part, with one web being arranged above and one web below the bore and/or that the cone surface has at least four vertical webs which extend along the cone surface in each case next to outlet openings of the bore, with the webs being straight or curved and in particular being arranged in a semicircular manner around the bore and preferably ends of the vertical webs coinciding in each case with a horizontal web. These webs on the conical surface of the plug can create a sufficiently tight seal between the plug and the housing. However, it is essential that the sealing surfaces of the webs have the necessary precision. However, it is also conceivable that the webs described are not arranged on the conical surface of the conical part, but are located on the inner wall of the housing. This makes it possible to create the same sealing connection between the plug and the housing and at the same time realize the manufacturing process described.

In particular, it is intended that the plug is manufactured in one piece with the cone part and the handle part from the same material. This material can be a plastic and preferably PEEK. This material is particularly easy to machine and also has the necessary hardness.

A method for solving the problem is described by the measures of claim 5. Accordingly, it is provided that a plug for a stopcock according to at least one of claims 1 to 3 is manufactured in one piece with the cone part and the handle part. This one-piece production simplifies the entire production process and makes it more cost-effective. The invention provides for the cone part and the handle part to be manufactured from a single workpiece by means of a turning and milling process. This combined process is carried out in a corresponding machine, namely a CNC machine. This one-piece production eliminates the need to join individual components. In addition, the turning and milling process allows the necessary precision to be achieved that is required for the tightness of the plug or stopcock.

In addition, the invention may provide for the shape of the cone part and the shape of the handle part to be reduced to essential shapes and features so that the plug can be manufactured in one piece by the combined turning and milling process. This preparatory or adapted design makes it possible to manufacture the plug in one piece. Accordingly, the shapes are selected in such a way that they are compatible with both the turning process and the milling process. It is conceivable that more complex structures of the cone surface are dispensed with in order to keep the cone surface simple, which also simplifies the turning process. It is conceivable that the more complex features, such as webs, which contribute to a sealing effect between the cone part and the housing, could be moved into the housing. This design of the plug, in particular the cone part and the handle part, contributes significantly to making the production of the plug particularly time and cost efficient.

A particularly advantageous embodiment of the invention may provide for the plug to be made of polyoxymethylene (POM). POM can be machined particularly well, whether by turning or milling or drilling. The use of this material enables an excellent surface quality after machining.

A solution to the said problem is described by the features of claim 8. Accordingly, it is provided that the plug, consisting of a cone part and a handle part, is formed in one piece. This plug can be rotatably mounted in an interior of a housing of a stopcock and has the cone part, which has a conical surface, and the handle part, which has a handle, lying one behind the other in the direction of an axis of rotation. The conical part also has a bore perpendicular to the axis of rotation, which can be designed as a fluid channel, whereby the conical surface can be brought into a sealing connection with an inner surface of the interior of the housing. A key feature is that the plug is designed as a disposable part, i.e. it is disposed of after a single use. This means that the entire manufacturing process can be made much simpler and material can be used that is much cheaper than the usual materials, which have to withstand high mechanical, chemical and thermal loads. Accordingly, this plug, which is designed as a disposable part, proves to be considerably cheaper to manufacture. The plug only has to withstand the mechanical stresses that occur during use for one application. It is therefore conceivable that relatively soft and inexpensive materials are used to manufacture the plug. However, it is also conceivable that a compostable plastic is used as the material, which can be disposed of in an environmentally friendly manner after use.

Preferably, it is further conceivable that the cone surface has at least two horizontal webs which extend in a ring-like manner around the cone part, with one web being arranged above and one web below the bore and/or that the cone surface has at least four vertical webs which extend along the cone surface in each case next to outlet openings of the bore, with the webs being straight or curved and in particular being arranged in a semicircular manner around the bore and preferably ends of the vertical webs coinciding in each case with a horizontal web. These webs on the conical surface of the plug can create a sufficiently tight seal between the plug and the housing. However, it is essential that the sealing surfaces of the webs have the necessary precision. Similarly, it is also conceivable that the webs described are not arranged on the conical surface of the conical part, but are located on the inner wall of the housing. This makes it possible to create the same sealing connection between the plug and the housing and at the same time realize the manufacturing process described.

A particularly advantageous embodiment of the invention can provide for the cone part, the handle part and, in particular, the bars to be manufactured in one piece from the same plastic. This design makes the plug particularly inexpensive to manufacture.

A particularly advantageous embodiment of the invention may provide for the plug to be made of polyoxymethylene (POM).

A stopcock for solving the said problem has the features of claim 12. Accordingly, it is provided that the stopcock has a plug according to at least one of claims 6 to 8 and a housing. The housing of the stopcock is designed as a reusable part. Accordingly, while the plug is replaced by a new plug after each application, the housing is subjected to a cleaning process in order to be ready for use for a further application. The housing must be able to withstand the mechanical, chemical and thermal stresses during cleaning. To ensure this, the housing is made of a suitable material, which also ensures that the housing, together with the disposable plug, has the required fluid tightness. This stopcock is particularly advantageous as it eliminates the need for very time-consuming and therefore cost-intensive preparation of the plug. In addition, a high level of patient safety can be achieved by using unused plugs. As the plug is made of a much softer material than the housing, which is preferably made of metal, the housing also proves to be much more durable, as there is little or no wear and tear.

A method for solving the said problem is described by the measures of claim 13. Accordingly, it is provided that a plug for a stopcock according to at least one of claims 6 to 8 is manufactured with the cone part and the handle part as a disposable part. Due to this design of the plug as a disposable part, the entire production can be simplified and made more cost-effective.

A particularly advantageous embodiment of the invention may provide for the plug to be made of polyoxymethylene (POM).

It is preferable for the cone part, the handle part and, in particular, the bars to be manufactured in one piece from the same plastic. A turning and milling, casting or injection molding process is a possible manufacturing process. However, it is also conceivable that the disposable plug is manufactured using a different process.

The drawing schematically illustrates a possible embodiment of the invention. It shows:

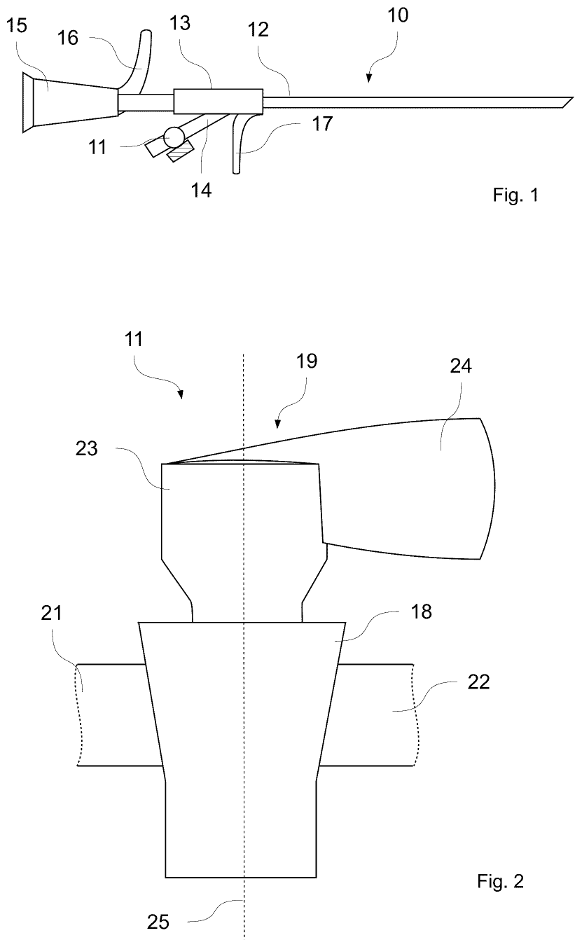

FIG. 1 a schematic side view of an endoscope with a stopcock,

FIG. 2 a schematic side view of a stopcock,

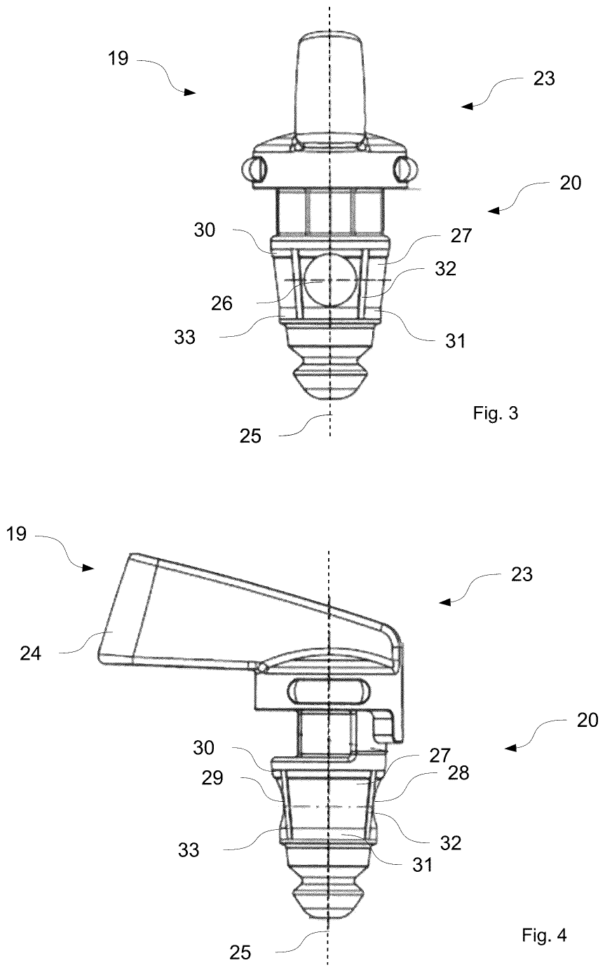

FIG. 3 a representation of a plug, and

FIG. 4 a further representation of the plug according to FIG. 3.

FIG. 1 shows a highly schematized endoscope 10. This endoscope 10 has a stopcock 11. Inside a shaft part 12 of the endoscope 10 runs a fluid channel, not visible here, which leads through a main body 13 at an angle to an inlet nozzle 14. The stopcock 11 is arranged at the inlet nozzle 14 to regulate the flow of liquid through the liquid channel. For example, a pump can be connected to the inlet nozzle 14 to supply liquid to the liquid channel.

The side view shown here shows the medical endoscope 10 in the orientation in which it is usually used during an operation. The endoscope 10 has an eyepiece 15 at its proximal end for observing the surgical site. To enable the surgeon to hold the endoscope 10 securely during the operation, two handles 16, 17 are attached to the endoscope 10. The handles 16, 17 are usually ergonomically shaped in such a way that the surgeon can hold the endoscope 10 in one hand with the aid of the handles 16, 17. The surgeon has the other hand free, for example to insert and operate through-hole instruments in a working channel of the instrument.

FIG. 2 shows a highly schematized representation of a possible embodiment of the stopcock 11 according to the invention. This stopcock 11 has a housing 18 and a plug 19, which is accommodated in the housing 18 with its conical part 20. The housing 18 has two connecting parts 21, 22 with which it can be coupled to the inlet connection 14 of the endoscope 10. These two connecting parts 21, 22 can also be associated with connecting parts, such as further valves or the like.

As can be seen in FIGS. 2 to 4, the plug 19 has a handle 23 along an axis of rotation 25 of the stopcock 11 above the cone part 20, which comprises a handle part 24. The handle part 24 can be ergonomically shaped for this purpose in order to facilitate holding and moving by the user.

According to the invention, the plug 19 is formed in one piece, i.e. the cone part 20 and the handle 23 are an integral part of the plug 19. Thus, the plug 19 described here proves to be advantageous compared to the known plugs, since it cannot be assembled from several components, for example by gluing. On the one hand, this reduces the assembly effort and, on the other hand, the one-piece design increases the stability of the plug 19.

By actuating the handle 23 or rotating the plug 19 about the axis of rotation 25, a fluid connection can be released or disconnected by the stopcock 11. For this purpose, the cone part 20 has a bore 26 which extends through the entire cone part 20 perpendicular to the axis of rotation 25. The bore 26 has two openings 28, 29, which are located in a conical surface 27 of the cone part 20. In the open position of the stopcock 11, the openings 28, 29 of the plug 19 are congruent with the tubular connection parts 21, 22 of the housing 18. To separate the fluid channel, the plug 19 is rotated about the axis of rotation 25 in such a way that the two openings 28, 29 of the bore 26 no longer overlap.

To ensure that, in the open position of the fluid channel, the fluid only passes through the bore 26 and not between the conical surface 27 and the wall of the interior of the housing 18 or that the stopcock 11 is actually fluid-tight in the closed position, the conical surface 27 has webs 30, 31, 32. In the embodiment example shown here, the conical surface 27 above and below the bore 26 are each assigned a ring-like horizontal web 30, 31. Due to the conical shape, the upper web 30 has a larger diameter than the lower web 31. In the embodiment example of the plug 19 shown in FIGS. 3 and 4, four vertical webs 32 are associated with these two webs 30, 31. The ends of the vertical webs 32 extend onto the horizontal webs 30, 31, so that the webs 30, 31, 32 have a common sealing surface 33. This sealing surface 33 is such that it coincides with the inner wall of the housing 18 and thus forms a sealing effect. The other features of the plug 19 visible in FIGS. 3 and 4 will not be discussed further here, as they are not relevant to the present invention.

### LIST OF REFERENCE SYMBOLS

10 Endoscope
11 Stopcock
12 Shaft part
13 Main body
14 Inlet connection
15 Ocular
16 Handle
17 Handle
18 Housing
19 Plug
20 Cone part
21 Connecting part
22 Connecting part
23 Handle
24 Handle part
25 Axis of rotation
26 Bore
27 Cone surface
28 Opening
29 Opening
30 horizontal bar
31 horizontal bar
32 vertical bar
33 Seal surface

1. Plug for a stopcock for producing and separating a fluid connection between at least two connecting parts, wherein the plug can be rotatably mounted in an interior of a housing of the stopcock and has a conical part having a conical surface and a handle part having a handle, lying one behind the other in the direction of rotation, wherein the plug is formed in one piece with the cone part and the handle part and wherein the cone part has a bore perpendicular to the axis of rotation and wherein the cone surface has webs and can be brought into a sealing connection with an inner surface of the interior of the housing, wherein the cone part and the handle part are produced from a workpiece by means of a turning and milling process.

2. Plug for a stopcock according to claim 1, wherein the shape of the cone part and the shape of the handle part are reduced to essential shapes which can be produced by a turning and milling process.

3. Plug for a stopcock according to claim 1, wherein the plug is made of polyoxymethylene.

4. Plug for a stopcock according to claim 1, wherein the cone surface has at least two horizontal webs which extend in a ring-like manner around the cone part, one web being arranged above and one web being arranged below the bore, and/or wherein the cone surface has at least four vertical webs, which extend along the conical surface in each case next to outlet openings of the bore, the webs being of straight or curved design and being arranged in a semicircular manner around the bore and ends of the vertical webs coinciding in each case with a horizontal web.

5. Method for producing a plug for a stopcock according to claim 1 for producing and separating a fluid connection between at least two connecting parts, wherein the plug can be rotatably mounted in an interior of a housing of the stopcock and has a conical part having a conical surface and a handle part having a handle lying one behind the other in the direction of an axis of rotation, wherein the plug is produced in one piece with the cone part and the handle part and wherein the cone part has a bore perpendicular to the axis of rotation, and wherein the cone surface can be brought into a sealing connection with an inner surface of the interior of the housing, wherein the cone part and the handle part are produced from a workpiece by means of a turning and milling process.

6. Method for producing a plug according to claim 5, wherein the shape of the cone part and the shape of the handle part for the turning and milling process are reduced to essential shapes.

7. Process for producing a plug according to claim 5, wherein the plug is produced from polyoxymethylene.

8. Plug for a stopcock for producing and separating a fluid connection between at least two connecting parts, wherein the plug can be rotatably mounted in an interior of a housing of the stopcock and has a conical part having a conical surface and a handle part having a handle lying one behind the other in the direction of an axis of rotation, wherein the plug is formed in one piece with the cone part and the handle part and wherein the cone part has a bore perpendicular to the axis of rotation and wherein the cone surface has webs and can be brought into a sealing connection with an inner surface of the housing, wherein the plug is formed as a disposable part.

9. Plug for a stopcock according to claim 8, wherein the cone surface has at least two horizontal webs which extend in a ring-like manner around the cone part, one web being arranged above and one web being arranged below the bore and/or wherein the cone surface has at least four vertical webs, which extend along the conical surface in each case next to outlet openings of the bore, the webs being straight or curved and being arranged in a semicircular manner around the bore and ends of the vertical webs coinciding in each case with a horizontal web.

10. Plug for a stopcock according to claim 8, wherein the cone part, the handle part and the webs are made in one piece from the same plastic.

11. Plug for a stopcock according to claim 8, wherein the plug is made of polyoxymethylene.

12. Stopcock for creating and separating a fluid connection between at least two connecting parts with a plug according to claim 8 and a housing, wherein the housing is designed as a reusable part.

13. Method for producing a plug for a stopcock according to claim 8 for producing and separating a fluid connection between at least two connecting parts, wherein the plug can be rotatably mounted in an interior of a housing of the stopcock and has a conical part having a conical surface and a handle part having a handle lying one behind the other in the direction of an axis of rotation, wherein the plug is produced in one piece with the cone part and the handle part and wherein the cone part has a bore perpendicular to the axis of rotation and wherein the cone surface has webs and can be brought into a sealing connection with an inner surface of the housing, wherein the plug is produced as a disposable part.

14. Method for producing a plug for a stopcock according to claim 13, wherein the cone part, the handle part and the webs are produced in one piece from the same plastic.

15. Method for manufacturing a plug for a stopcock according to claim 13, wherein the plug is made of polyoxymethylene.

16. Method for producing a plug for a stopcock according to claim 13, wherein the cone part is produced in one piece with the handle part by a turning and milling, casting or injection moulding process.
